(19) United States
(12) Patent Application Publication
(10) Pub. No.: US 2007/0083151 A1
(43) Pub. Date: Apr. 12, 2007

(54) Medical devices and kits including same

(76) Inventor: Chad J. Carter, Lake Elmo, MN (US)

Correspondence Address: 3M INNOVATIVE PROPERTIES COMPANY, PO BOX 33427, ST. PAUL, MN, 55133-3427, US

(21) Appl. No.: 10/596,874

(22) PCT Filed: Dec. 14, 2004
(86) PCT No.: PCT/US04/41732

§ 371 Date: Jun. 28, 2006

Related U.S. Application Data
(60) Provisional application No. 60/532,987, filed on Dec. 29, 2003.

(51) Int. Cl. A61B 17/20

(52) U.S. Cl. 604/46

(57) ABSTRACT

A medical device is described, comprising an array (10) comprising microstructures (12) configured to penetrate the stratum corneum upon impact, and a connection member (20) affixed to the array in a one piece construction, the connection member configured to reversibly connect the medical device to an applicator. The medical devices of the invention may be used in methods requiring the penetration of skin to deliver medicaments or other substances and/or extract blood or tissue through the skin. In use, it is generally desirable to provide the microstructures at a height sufficient to penetrate the stratum corneum. A medical kit is also described, comprising the foregoing medical device and a tray (34) configured to hold the medical device.

The present invention relates to medical devices and to kits that incorporate the medical devices.

### BACKGROUND OF THE INVENTION

Some therapeutic molecules can be delivered to the body through the skin. The main barrier to the transport of such molecules through the skin is the outermost layer of the skin known as the stratum corneum. Devices that include a plurality of piercing elements are often referred to as microneedles, microneedle arrays, micro arrays, micro-pins or the like. These medical devices can pierce the stratum corneum by making a plurality of microscopic slits in the outermost layer of skin to facilitate the transdermal delivery of therapeutic agents or the sampling of fluids through the skin. The microneedle devices are pressed against the skin to pierce the stratum corneum and thereby permit the delivery of a therapeutic agent through the stratum corneum and into the tissue below or to permit the sampling of transdermal body analytes as they exit the body through the microscopic slits.

Micro arrays may be used in conjunction with an applicator device capable of being used a number of different times. Unlike the applicator, the micro arrays are generally used once and then disposed of. The arrays are typically manufactured in a flat sheet-like configuration and temporarily attached to the applicator using, for example, an adhesive that permits the array to be detached from the applicator using a tweezers or the like. The process of removing and replacing an array can be very tedious and slow and may create a risk of damaging the numerous structures in the array. Additionally, the process of removing the array from the applicator requires a technician or other person to handle and dispose of the used array, creating handling issues to ensure that the risk of infection is minimized.

There is a need for a micro array that can be attached to an applicator prior to use and then detached from the applicator after use without the need to handle the array directly. There is also a need to provide a micro array in a construction that avoids the need for adhesives and the requirement to use tweezers or similar tools in the attachment/detachment process. It is desirable to provide a microneedle array in a one-piece construction that includes an array with means for reversibly attaching the array to an applicator. It is also desirable to provide a kit that facilitates the attachment of micro arrays to an applicator to facilitate use of the arrays.

### SUMMARY OF THE DISCLOSURE

In one aspect, the invention provides a medical device, comprising: an array comprising microstructures configured to penetrate the stratum corneum upon impact; and a connection member affixed to the array in a one piece construction, the connection member configured to reversibly connect the medical device to an applicator.

In another aspect, the invention provides a medical kit, comprising: the medical device as described herein; and a tray configured to hold the medical device.

As used herein, certain terms will be understood to have the meaning set forth below:

“Array” refers to the medical devices described herein that include one or more structures capable of piercing the stratum corneum to facilitate the transdermal delivery of therapeutic agents or the sampling of fluids through the skin.

“Microstructure” or “micro array” refers to the specific microscopic structures associated with the array that are capable of piercing the stratum corneum to facilitate the transdermal delivery of therapeutic agents or the sampling of fluids through the skin. By way of example, microstructures can include needle or needle-like structures as well as other structures capable of piercing the stratum corneum.

The features and advantages of the present invention may be more broadly and clearly understood by those skilled in the art upon consideration of the remainder of the disclosure including the Detailed Description of the Preferred Embodiment as well as the appended claims.

### BRIEF DESCRIPTION OF THE DRAWINGS

In the description of the preferred embodiment, reference is made to the various Figures, wherein:

FIG. 1 is a perspective view of an array according to the present invention;

FIG. 2 is another perspective view of the array shown in FIG. 1;

FIG. 3 is a perspective view of another embodiment of an array according to the present invention;

FIG. 4 is a perspective view of still another embodiment of an array according to the present invention;

FIG. 5 is a perspective view of a kit according to the invention; and

FIG. 6 is a side elevation, shown in cross section, of a portion of the kit shown in FIG. 5.

### DETAILED DESCRIPTION OF THE PREFERRED EMBODIMENT

The invention provides an array useful in the delivery of therapeutic agents or in the sampling of fluids through the stratum corneum. The array of the invention is provided in a one-piece construction that facilitates the attachment of the array to an applicator as well as facilitating the detachment of the array from the applicator after use. The invention also provides a kit that includes the foregoing array, The kit facilitates attachment of the array to an applicator while also providing the array in a ready-to-use condition. In the description of the preferred embodiment, reference is made to the various Figures, wherein reference numerals are used to identify certain structures in the embodiments and wherein like reference numerals indicate like structures.

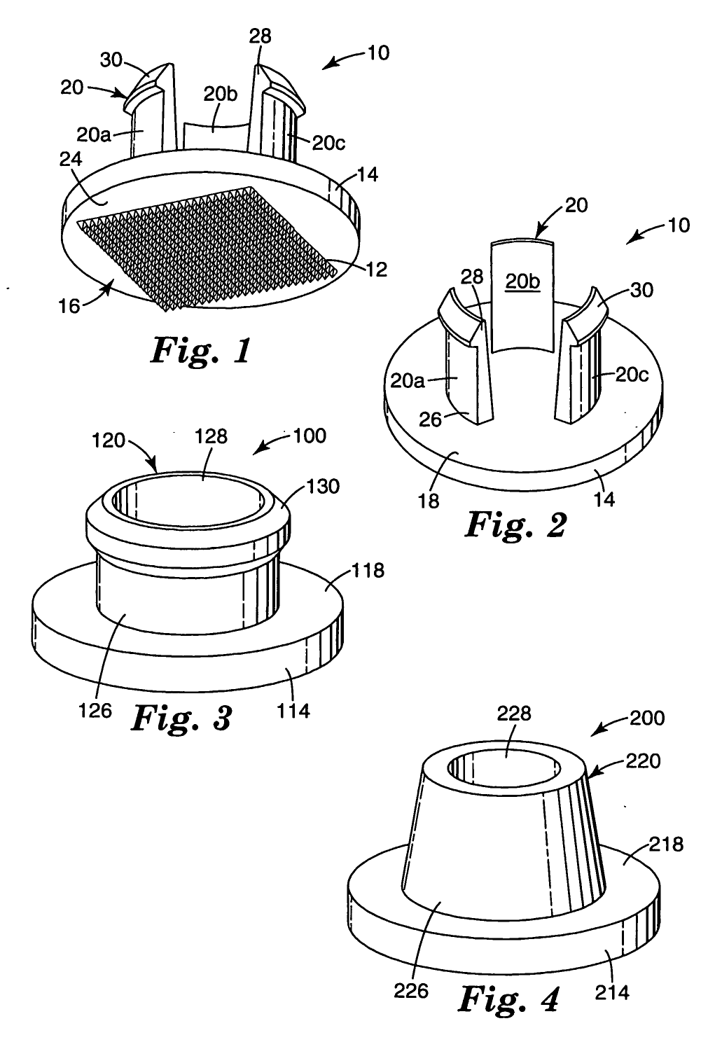

Referring now to the Figures, FIGS. 1 and 2 illustrate a medical device according to an embodiment of the invention in the form of an array 10. The array 10 includes a plurality of microstructures 12 capable of puncturing the stratum corneum when applied against the surface of the skin. The microstructures are affixed to a collar 14 having first and second major surfaces 16 and 18, respectively. The microstructures 12 are affixed to and arranged on the first major surface 16 of the collar 14, and a connection member 20 is affixed to and extends from the second major surface 18 of collar 14. The first major surface 16 of the collar 14 includes a first zone commensurate in area with the microstructures 12 and extending outwardly from the centermost portion of the first major surface 16 of the collar 14. A second zone 24 extends from the outer edge of the microstructures 12 in the first zone to the outermost edge of the first major surface 16. The second zone 24 is provided with no structures thereon, thus preserving an essentially planar portion along the outermost periphery of the first major surface 16. The utility of the second zone 24 on the first major surface 16 of collar 14 is described below.

As shown, the array 10 is provided in a one-piece construction. The connection member 20 provides a means for the attachment of the array 10 to an applicator device (not shown). The connection member 20 is affixed to the second major surface 18 of the collar 14 with the connection member extending away from the second major surface 18. In the depicted embodiment, the connection member 20 includes a plurality of leg members 20a, 20b, and 20c, each having first ends 26 affixed to the second major surface 18 of the collar 14 and second ends 28 disposed remotely from the second major surface. Each of the leg members extends in an essentially perpendicular relationship to the second major surface 18. Each of the second ends 28 include a lip 30 that is configured to engage with a mating slot (not shown) on an applicator to provide a snap-fit that reversibly retains the array 10 within an applicator until the array 10 is intentionally released. Applicator constructions suitable for use in conjunction with the arrays of the present invention are described, for example, in co-pending U.S. patent application, Ser. No. 10/621,620, filed on Jul. 17, 2003.

In this arrangement of parts, the connection member 20 comprises a plurality of three leg members 20a, 20b, and 20c capable of flexing under pressure along the attachment line between each leg and the second major surface of the collar 14. The connection members may be inserted into mating portions of the applicator by flexing the three leg members 20a, 20b, and 20c inwardly so that their respective second ends 28 are flexed toward one another while the connection member is inserted into an applicator. The lips 30 will each be received within a compatible or mating slot within the applicator to achieve a “snap-fit” connection that reversibly retains the array with the applicator. Likewise, the three legs 20a, 20b, and 20c can again be compressed in a known manner to disengage the snap-fit connection and allow for the release of the array 10 from the applicator. The mechanical principles of a snap-fit connection, as described herein, are well within the knowledge of those practicing in the field.

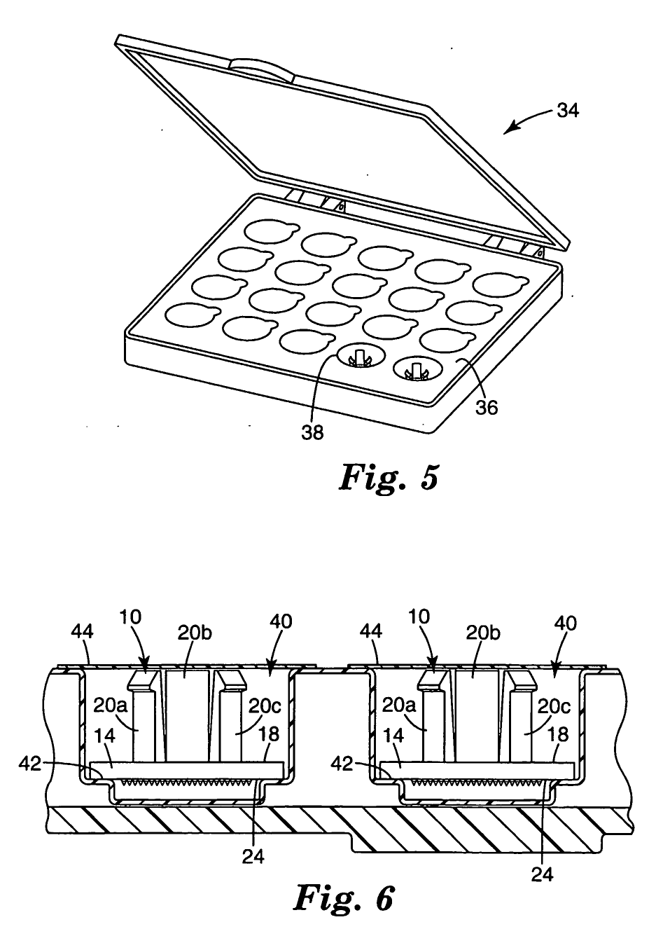

The present invention also provides a kit that includes the above described medical device or array along with a tray that retains and stores the array for use when it is needed. As contemplated herein, the tray is configured to store and provide at least one array and typically a plurality of arrays in a ready-to-use condition. Referring now to FIGS. 5 and 6, one embodiment of a tray 34 is illustrated and will now be described. The tray 34 comprises a component of a kit according to the present invention. The tray 34 includes a first surface 36 and a plurality of openings 38 therein. The openings 38 provide access to a plurality of wells 40 with each individual well being associated with one of the openings 38. The first surface 36 of the tray 34 is substantially planar with each of the wells 40 extending in the same direction from the openings 38 and away from the first surface 36.

Each well 38 is configured to hold and retain one array such as the array 10 previously described herein. Well 40 is dimensioned so that the full length or height of the array 10 is retained within the well 40 with all parts of the array 10 situated below the first surface 36 of the tray 34. A shoulder 42 in the inner periphery of the well 40 is provided to support the array 10 with the shoulder 42 resting against the second zone 24 on the first major surface 16 on collar 14. The array 10 may be positioned within the well 40 with the second zone 24 of the first major surface 16 simply resting against the shoulder, or the inner diameter of the well 40 may provide a friction fit against the outer diameter of the collar 14. The array 10 is positioned within the well so that the connection member 20 on the array 10 is nearest the opening 38.

In an alternate embodiment, the foregoing array 10 may be supported within the well 40 by a friction fit such that the shoulder 42 is not needed. In such an embodiment, the microstructures 12 are positioned in a first zone that extends across the entire first major surface 16 of the collar 14, such that the first major surface 16 includes no area free of microstructures comparable to the second zone 24. Alternatively, the shoulder 42 may be replaced by a central pillar extending from the base of the well and supporting the array 10. In such an embodiment, a second zone may be provided free of micro array structures in a central portion of the first major surface 16 of the collar 14 with the second zone surrounded by a first zone comprising the microstructures such as microstructures 12.

In the described construction, the tray 34 and the array 10 are both typically provided in a disinfected condition. A sealing member (not shown) such as a polymer membrane or film may be disposed over the openings 38 in tray 34 to hermetically seal the array 10 within the well 38. The sealing membrane or film may be affixed to the first surface 36 of the tray 34 with an appropriate adhesive or the like to preferably provide a hermetic seal. In this construction, the array 10 within well 38 may be provided in a ready-to-use condition (e.g., without the need for further disinfection). When the array 10 is to be used, the membrane or film covering the well 38 is simply removed (e.g., by peeling it away from the opening 38) or it may be cut, broken, or pierced to allow the insertion of an applicator into the well 38 to engage the connection member 20 and retrieve the array 10 from the tray 34.

Other embodiments are contemplated within the scope of the invention, including other configurations for the array of the invention. Such alternate embodiments will now be described with the understanding that all of the arrays described herein have certain similarities to one another, and the features of the different embodiments of the arrays that are identical to one another will not be repeated.

Referring to FIG. 3, another embodiment of an array 100 is shown having a connection member 120 having a unitary concentric structure with its first end 126 affixed to the second major surface of the collar 114 and extending away from the collar to a second end 128. A single concentric lip 130 is provided around the second end 128 of the connection member 120 with the lip 130 provided in a configuration capable of being releasably retained within a complementary slot (not shown) on an applicator to provide a snap-fit connection between the array 100 and the applicator. Other than the above described features, the array 100 is substantially identical to the array 10 shown in FIGS. 1 and 2. It will be appreciated that the unitary construction for the connection member 120 will normally be somewhat less flexible than the connection member 20 of the array 10. Consequently, any flexibility that may be needed to provide a snap-fit between the connection member 120 and an applicator can be accounted for in the manufacture of the array 100 by, for example, the appropriate selection of materials. Alternatively, the structure of the applicator can be made to permit the flexibility needed to capture and subsequently release the lip 130.

Referring now to FIG. 4, still another embodiment of an array 200 is shown having a connection member 220 comprising a unitary concentric structure having its first end 226 affixed to the second major surface of the collar 214 with the connection member 220 extending away from the collar and terminating in a second end 228. The connection member 220 has a profile that tapers slightly as it extends from its first end 226 on the second major surface of collar 214 to its second end 228 with the outer diameter of connection member 220 at its first end 226 being greater than the outer diameter at its second end 228. Moreover, the connection member 220 includes a relatively smooth outer surface 221 that includes no pronounced protrusions or other structures for retaining the array 200 when it is held by an applicator. The connection member 220 is configured to be retained by a complementary attachment portion on an applicator using a friction fit to releasably retain the array 200.

The micro arrays useful in the various embodiments of the invention may comprise any of a variety of configurations. One embodiment for the micro arrays comprises the structures disclosed in United States patent application publication no. US2003/0045837. The disclosed microstructures in the aforementioned patent application are in the form of microneedles having tapered structures that include at least one channel formed in the outside surface of each microneedle. The microneedles may have bases that are elongated in one direction. The channels in microneedles with elongated bases may extend from one of the ends of the elongated bases towards the tips of the microneedles. The channels formed along the sides of the microneedles may optionally be terminated short of the tips of the microneedles. The microneedle arrays may also include conduit structures formed on the surface of the substrate on which the microneedle array is located. The channels in the microneedles may be in fluid communication with the conduit structures. Another embodiment for the micro arrays comprises the structures disclosed in co-pending U.S. patent application, Ser. No. 10/621,620 filed on Jul. 17, 2003 which describes microneedle devices with microneedles having a truncated tapered shape and a controlled aspect ratio. Still another embodiment for the micro arrays comprises the structures disclosed in U.S. Pat. No. 6,091,975 (Daddona, et al.) which describes blade-like microprotrusions for piercing the skin. Still another embodiment for the micro arrays comprises the structures disclosed in U.S. Pat. No. 6,313,612 (Sherman, et al.) which describes tapered structures having a hollow central channel. Still another embodiment for the micro arrays comprises the structures disclosed in International Publication No. WO 00/4766 (Garstein, et al.) which describes hollow microneedles having at least one longitudinal blade at the top surface of tip of the microneedle.

The arrays of the invention may be manufactured in any manner that provides a unitary construction. Typically, the arrays or the invention are molded one-piece medical devices made from one or more polymeric materials, and often using the same polymeric material to mold all of the foregoing features of the array in a unified and unitary construction. It will be appreciated, however, that the different portions of the array may comprise different material(s) as may be required to impart rigidity to portions of the array while allowing other portions of the array to be manufactured with less rigid or more flexible material.

Suitable materials for the manufacture of the arrays of the invention include those that are moldable (by, e.g., injection molding, compression molding, etc.), have a high modulus of elasticity, and high elongation at break. Suitable polymeric materials for the arrays of the present invention include acrylonitrile-butadiene-styrene (ABS) polymers, polyphenyl sulfides, polycarbonates, polypropylenes, acetals, acrylics, polyetherimides, polybutylene terephthalates, polyethylene terephthalates as well as other known materials. A suitable method for molding the arrays of the invention is described in patent application Ser. No. 60/546,780.

Similarly, the trays used in the kits of the present invention may be suitably manufactured by any of a variety of manufacturing methods using any of a variety of materials. Typically, the trays are molded articles made from one or more polymeric materials, and typically using the same polymeric material for all of the foregoing features of the tray. However, different portions of the tray may comprise different material(s) as may be required to impart rigidity to portions of the tray while allowing other portions of the tray to be manufactured with less rigid or more flexible materials.

Suitable materials for the manufacture of the trays to be used in the kits of the invention include those that are moldable (by, e.g., injection molding, compression molding, etc.), have a high modulus of elasticity, and high elongation at break. Suitable polymeric materials for the trays of the present invention include acrylonitrile-butadiene-styrene (ABS) polymers, polyphenyl sulfides, polycarbonates, polypropylenes, acetals, acrylics, polyetherimides, polybutylene terephthalates, polyethylene terephthalates as well as other known materials.

The microneedle arrays of the invention may be used in a variety of different manners. One manner of using microneedle arrays of the present invention is in methods involving the penetration of skin to deliver medicaments or other substances and/or extract blood or tissue through the skin. In use, it is generally desirable to provide the microstructures of the array at a height sufficient to penetrate the stratum corneum. When delivering a medicament or therapeutic agent, the agent may be applied directly to an area of the skin and the array is then applied to the same area of the skin by contacting the skin with the microstructures of the array with sufficient force to puncture the stratum corneum and thereby allow the therapeutic agent to enter the body through the outermost layer of the skin. Alternatively, the array may be applied to an area of the skin to puncture the stratum corneum and the medicament or therapeutic agent applied to the same area of the skin (e.g., in the form of a cream, gel, or adhesive bandage). In a third alternative, the medicament may be applied to the microstructured area of the array in the form of a coating. The array is contacted with the skin with sufficient force to puncture the stratum corneum. The medicament coated on the microstructured area of the array may then be mechanically deposited into the skin tissue or dissolved from the array by body fluids allowing the medicament to be absorbed into the skin tissue. The parameters for the delivery of therapeutic agents using the medical devices of the invention are suitably described in the aforementioned co-pending patent applications, Ser. Nos. 09/947195 and 10/621620.

It will be appreciated by those skilled in the art that the foregoing detailed description is not to be construed as limiting the ultimate configuration of the medical devices (e.g., the arrays) or the medical kits of the present invention. The described embodiments, while exemplary of structures contemplated as being within the scope of the invention, are not exhaustive. For example, the described connection member may comprise any configuration reasonably capable of being adapted for connecting the array to an applicator. Accordingly, the connection member may comprise a single unitary structure of the types already described and variations thereof, or the connection member may comprise a multi-piece or multi-member construction that includes two, three, four or any number of members. Additionally, the means for connecting the arrays of the invention to an applicator are not to be unduly limited to the snap fit or friction fit embodiments described herein. Rather, the invention contemplates any means for the reversible connection of an array to an applicator by mating complementary parts in a connection scheme that permits a quick and secure connection so that the array may be used in, for example, the application of a therapeutic agent while also allowing for the easy release of the array from the applicator when desired by a user of the device.

None of the various Figures described herein are to be assumed to constitute scale drawings, and the invention is not to be construed as limited in its physical dimensions to the various embodiments discussed and described herein or depicted in the various Figures. Insubstantial variations of the embodiments described and claimed herein may be possible. All such variations, including those that are unforeseeable at this time by those reasonably skilled in the art, are to be considered within the scope of the present invention.

What is claimed:

1. A medical device, comprising:
An array comprising microstructures configured to penetrate the stratum corneum upon impact;
A connection member affixed to the array in a one piece construction, the connection member configured to reversibly connect the medical device to an applicator.

2. The medical device as in claim 1, the array further comprising a collar having first and second major surfaces, the microstructures disposed on and affixed to the first major surface and the connection member disposed on and affixed to the second major surface.

3. The medical device as in claim 2, wherein the microstructures comprise a plurality of identically configured members.

4. The medical device as in claim 3, wherein the microstructures are in a circular array.

5. The medical device as in claim 4 wherein the collar is circular and the first major surface comprises a first zone extending from the center of the first major surface, the microstructures disposed within the first zone, and a second zone extending from the first zone to the periphery of the first major surface, the second zone being free of the microstructures.

6. The medical device as in claim 1 wherein the connection member comprises a plurality of leg members having first and second ends thereon, the first ends of the leg members affixed to the array and the second ends of the leg members configured to reversibly connect the medical device to an applicator.

7. The medical device as in claim 6 wherein the connection member comprises three leg members.

8. The medical device as in claim 1 wherein the connection member comprises a single structure having first and second ends thereon, the first end of the connection member affixed to the array and the second end of the connection member configured to reversibly connect the medical device to an applicator.

9. The medical device as in claim 1 wherein the connection member is configured to reversibly connect the device to an applicator by an interference fit.

10. A molded medical device comprised of molded polymeric material and constructed as in claim 1.

11. The molded device of claim 10 wherein the molded polymeric material is selected from the group consisting of polycarbonate, polyetherimide, polyethylene terephthalate, and blends thereof.

12. A medical kit, comprising:
The medical device of claim 1; and
A tray configured to hold the medical device.

13. The medical kit as in claim 12 wherein the tray comprises a first surface and at least one well configured to hold a medical device therein, the well comprising an opening in the first surface so that the medical device, when positioned within the well, is accessible through the opening in the first surface.

14. The medical kit as in claim 13 further comprising a plurality of wells, each well being similarly configured to hold a single medical device therein.

15. The medical kit as in claim 12 wherein the array of the medical device further comprises a collar having first and second major surfaces, the microstructures disposed on and affixed to the first major surface and the connection member disposed on and affixed to the second major surface.

16. The medical kit as in claim 15 wherein the first major surface of the collar comprises a first zone extending from the center of the first major surface, the microstructures disposed within the first zone, and a second zone extending from the first zone to the periphery of the first major surface, the second zone being free of the microstructures.

17. The medical kit as in claim 16 wherein the first surface of the tray defines a substantially planar surface and wherein the well further comprises a shoulder configured to rest against the second zone of the first major surface of the collar without engaging the microstructures in the first zone, the well extending away from the first surface of the tray.

18. The medical kit as in claim 17 wherein the tray further comprises a sealing member disposed over the opening in the first surface to hermetically seal the well when the medical device is retained therein, the sealing member being removable or breakable to permit withdrawal of the medical device from the well.
